SEQUENCE LISTING (8 sequences)

SEQ ID NO: 1 - DNA, 23 bp, Artificial Sequence
  PCR PRIMER
ttggtcatgatactgctgattgc

SEQ ID NO: 2 - DNA, 20 bp, Artificial Sequence
  PCR PRIMER
cgtcgtcgtgaccaactcta

SEQ ID NO: 3 - DNA, 24 bp, Artificial Sequence
  PCR PRIMER
cagaggagcacagtctcagtgttc

SEQ ID NO: 4 - DNA, 26 bp, Artificial Sequence
  PCR PRIMER
tctctgagagtgctgcttatctgtgt

SEQ ID NO: 5 - DNA, 24 bp, Artificial Sequence
  PCR PRIMER
acctccgaaactgaagatctccta

SEQ ID NO: 6 - DNA, 21 bp, Artificial Sequence
  PCR PRIMER
tgctggttgaagaatgcttga

SEQ ID NO: 7 - DNA, 20 bp, Artificial Sequence
  PCR PRIMER
aacaccccagccatgtacgt

SEQ ID NO: 8 - DNA, 24 bp, Artificial Sequence
  PCR PRIMER
cggtgaggatcttcatgaggtagt

(19) United States
(12) Patent Application Publication
(10) Pub. No.: US 2022/0323471 A1
(43) Pub. Date: Oct. 13, 2022

(54) GLYCOSYLATED DIPHYLLIN AS A BROAD-SPECTRUM ANTIVIRAL AGENT AGAINST ZIKA VIRUS AND COVID-19

(71) Applicant: ALBERT EINSTEIN COLLEGE OF MEDICINE, Bronx, NY (US)

(72) Inventor: Felipe DIAZ-GRIFFERO, New York, NY (US)

(21) Appl. No.: 17/638,606

(22) PCT Filed: Aug. 28, 2020
(86) PCT No.: PCT/US2020/048463

§ 371 Date: Feb. 25, 2022

Related U.S. Application Data
(60) Provisional application No. 62/894,032, filed on Aug. 30, 2019.

(51) Int. Cl. A61K 31/7048; A61K 31/365; A61P 31/14

(52) U.S. Cl. CPC A61K 31/7048; A61K 31/365; A61P 31/14

(57) ABSTRACT

The disclosure provides a method for preventing or treating a flavivirus infection, a filovirus infection, a SARS-CoV-1 infection, a SARS-CoV-2 infection, or a MERS-CoV infection, the method comprising administering to a subject in need thereof a therapeutically effective amount of a compound of Formula I or a pharmaceutically acceptable salt thereof. The disclosure also provides a method for preventing or treating a filovirus infection, a SARS-CoV-1 infection, a SARS-CoV-2 infection, or a MERS-CoV infection with a compound of Formula II or pharmaceutically acceptable salt thereof. The structures of Formula I and Formula II are shown below.

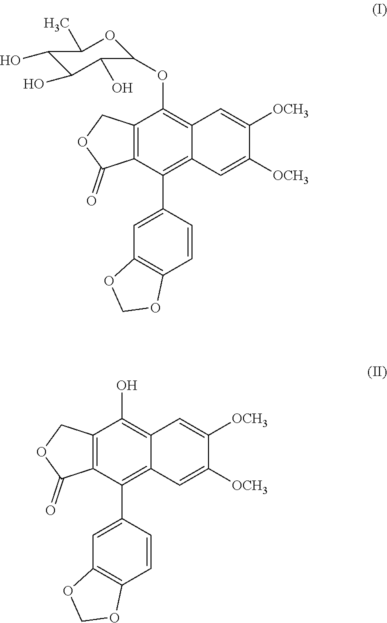 [SMILES: COc1cc2c(OC3OC(C)C(O)C(O)C3O)c3c(c(-c4ccc5c(c4)OCO5)c2cc1OC)C(=O)OC3 ; COc1cc2c(O)c3c(c(-c4ccc5c(c4)OCO5)c2cc1OC)C(=O)OC3]

### CROSS REFERENCE TO RELATED APPLICATION

This application claims priority of U.S. Provisional Appl. No. 62/894,032, filed Aug. 30, 2019, which is hereby incorporated by reference in its entirety.

### FIELD OF DISCLOSURE

The present disclosure relates methods of prevention or treatment of viral infections with pharmaceutical compositions containing glycosylated diphyllin and/or diphyllin.

### BACKGROUND

Zika virus (ZIKV) is a mosquito-borne flavivirus. Though ZIKV infection in humans usually results in mild symptoms, infection during pregnancy can result in serious birth defects, in particular microcephaly. ZIKV infection in adults sometimes results in serious Guillian-Barre syndrome, a serious autoimmune disorder affecting the nervous system. The Flavivirus genus, to which ZIKV belongs, also includes several other important vector-borne human pathogens such as the West Nile virus (WNV), dengue virus (DENV), tick-borne encephalitis virus (TBEV), and Japanese encephalitis virus (JEV). Some of these viruses are widespread in the equatorial region, where the mosquito vectors are most prevalent. Although ZIKV is known to be primarily transmitted through mosquito bites, some studies have shown that it can also be sexually transmitted. For these reasons, ZIKV was recognized in 2016 as a Public Health Emergency of International Concern (PHIC) by the World Health Organization (WHO).

Flaviviruses such as Zika cause sporadic pandemic outbreaks worldwide. There is an urgent need for anti-Zika virus (ZIKV) drugs to prevent mother-to-child transmission of ZIKV, new infections in high-risk populations, and the infection of medical personnel in ZIKV-affected areas.

Ebola virus (EBV) is a hemorrhagic fever that is often fatal to humans. EBV is a member of the Filoviridae family and filovirus genus. People become infected through contact with infected animals such as fruit bats, chimpanzees, gorillas, monkeys, forest antelope, or porcupines or through contact with the bodily fluids of an infected person. The current approach to prevent the spread of infection is to contain an outbreak and to prevent it from spreading by following infection control procedures. Some vaccines to protect against EBV are in development, however there are no drugs for the treatment of EBV.

Although the number of EBV outbreaks is limited, the average fatality rate is around 50%. In addition, health care workers have often become infected while treating those with EBV. Thus, there is a need for anti-EBV drugs to prevent EBV infection in those at risk of infection and to treat patients with EBV infection.

MERS-CoV (Middle East respiratory syndrome) is an infectious disease caused by a coronavirus. MERS-CoV was first reported in Saudi Arabia in 2012 and cases were confirmed in several other countries including the United States. Infection with MERS-CoV causes sever respiratory disease with symptoms including fever, cough, and shortness of breath. The mortality rate of MERS-CoV rate during the 2012 outbreak was approximately 35%, but since 2012 there have been very few cases. While MER-CoV is currently a very rare infection there remains a need for effective therapies in cases of future outbreaks.

SARS-CoV-1 (Severe acute respiratory syndrome coronavirus) is an infectious coronavirus that causes severe illness, with symptoms including muscle pain, headache, fever, and respiratory symptoms lasting 2-14 days. Respiratory symptoms include cough and pneumonia. The mortality rate for a 2003 outbreak was 9% overall and 50% in patients over 60. There are no available treatments for SAR-CoV-1, but there have been no outbreaks of SARS-CoV-1 since 2003.

SARS-CoV-2, (COVID-19), is a viral infectious disease first identified in China in 2019. SARS-CoV-2 is highly infectious and since its identification has caused a global pandemic resulting in 23 million confirmed infections and more than 800,000 confirmed deaths. Total infections and deaths are almost certainly higher. SARS-CoV-2 mortality rates vary by country with most countries reporting a mortality rate of 2-5%. Morality is higher in older patients and those with pre-existing conditions. While several drugs have been identified as effective for lessening the severity of symptoms and shortening hospital stays for severely afflicted patients, therapies that can be administered prophylactically to reduce the likelihood of infection or administered to mildly afflicted patients early in infection to decrease the length and severity of their symptoms, are still needed.

### SUMMARY

The disclosure provides a method for preventing or treating viral infection with a flavivirus, a Filovirus, SARS-CoV-1 virus, a SARS-CoV-2 (COVID-19) virus, or a MERS-CoV virus. The method comprises administering to a subject in need thereof a therapeutically effective amount of Formula I:

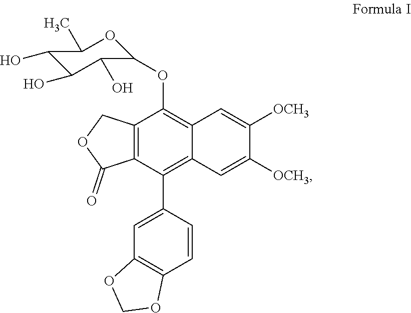 [SMILES: COc1cc2c(OC3OC(C)C(O)C(O)C3O)c3c(c(-c4ccc5c(c4)OCO5)c2cc1OC)C(=O)OC3]

or a pharmaceutically acceptable salt thereof.

The disclosure also provides a method for preventing or treating a flavivirus infection, a SARS-CoV-1 infection, a SARS-CoV-2 infection (COVID-19), or a MERS-CoV infection comprising administering to a subject in need thereof a therapeutically effective amount of compound of Formula II:

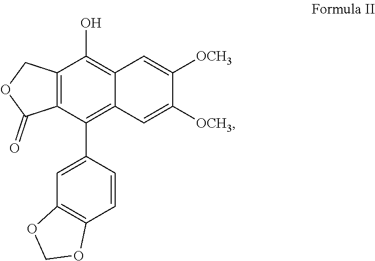 [SMILES: COc1cc2c(O)c3c(c(-c4ccc5c(c4)OCO5)c2cc1OC)C(=O)OC3]

or a pharmaceutically acceptable salt thereof.

The above described and other features are exemplified by the following figures and detailed description.

The compounds of Formula I and Formula II can be administered neat, or as part of a pharmaceutical composition that comprises a compound or salt of Formula I or a compound or salt of Formula II, together with a pharmaceutically acceptable excipient, and optionally with an active agent in addition to the compound or salt of Formula I or Formula II.

### BRIEF DESCRIPTION OF THE DRAWINGS

FIGS. 1A-1C show that DGP inhibits ZIKV infection of HT1080 (FIG. 1A), VERO (FIG. 1B), and CHME3 (FIG. 1C) cells when challenged with ZIKV-MR766 at a multiplicity of infection (MOI) of 1 in conjunction with increasing concentrations of DGP.

FIGS. 2A-2C show that DGP inhibits ZIKV infection of HT1080 (FIG. 2A), VERO (FIG. 2B), and CHME3 (FIG. 2C) cells when challenged with ZIKV-RVPs at a multiplicity of infection (MOI) of 1 in conjunction with increasing concentrations of DGP.

FIG. 3A: ZIKV RNA levels were measured using real-time PCR for HT1080 and VERO cells challenged by ZIKV MR766 at an MOI of 1 in the presence of DGP. ZIKA viral RNA levels were normalized to actin.

FIG. 3B: Similar infections were used to determine infectivity via flow cytometry using anti-4G2 antibodies.

FIGS. 4A-4C show the kinetics of ZIKV entry into the cell. ZIKV-RVPs were pre-bound to HT1080 cells at 4° C. for 1 hour. At the indicated time points, cells were treated with 1 μM of DGP (FIG. 4A), 1 μM of Nanchangmycin (FIG. 4B), and 20 mM ammonium chloride (NH₄Cl, FIG. 4C).

FIG. 5 shows the effect of 0.1 μM DGP, 1 μM DGP, and 20 mM NH₄Cl on the ability of ZIKV MR766 to infect CHME3 cells via the induction of IFN-β normalized to actin and the percentage of cells positive for anti-4G2 antibodies.

FIG. 6 shows the effect of 0.1 μM DGP, 1 μM DGP, and 20 mM NH₄Cl on the ability of SeV to infect CHME3 cells via the induction of IFN-β normalized to actin and the percentage of cells positive for anti-4G2 antibodies.

FIG. 7 shows that DGP blocks infectivity of other flaviviruses including DENV1, JEV, TBEV, WNV, as well as Filovirus EBV in VERO cells. This showed that DGP has broad-spectrum antiviral activity.

FIGS. 8A-8D demonstrate that DGP prevents ZIKV-induced mortality in type I Interferon receptor knockout mice (Ifnar1^{-/-}). FIG. 8A shows that the percent survival was improved for those groups receiving DGP at a 0.1 mg/kg and 0.2 mg/kg dose. FIG. 8C shows that increasing the dose of DGP to 1 mg/kg resulted in 100% survival. FIGS. 8B and 8D show that the surviving mice receiving DGP (0.2 mg/kg and 1 mg/kg, respectively) were able to regain lost body weight. Weights are expressed as percentage of body weight prior to infection, and standard deviations are shown.

FIGS. 9A-9B show the effect of DGP, diphyllin, and 6-deoxyglucose (6DG) on HT1080 cells (FIG. 9A) and CHME3 cells (FIG. 9B) infected with ZIKV MR766 at an MOI of 0.5. Specific IC₅₀ values for each molecule that inhibits ZIKV infection are shown. Bars represent the Mean±SD. P<0.05 (*), P<0.01 (**), P<0.001 (***), or not significant (ns), using two-tailed Student's t-test are shown.

FIG. 10A-10D show that DGP prevents the acidification of endosomes. FIG. 10A shows the effect of the controls Bafilomycin A (100 nM) and NH₄Cl (25 mM) on HT1080 cells. The effect on the acidification of HT1080 cells treated with the indicated concentrations of DGP, diphyllin, and 6DG is shown in FIG. 10B, FIG. 10C, and FIG. 10D, respectively. The fluorescence intensity of AO was measured by flow cytometry using the PerCP-Cy5-5-A (695 nm) channel. Changes in fluorescence are shown using histograms and the black arrow represents the shift in fluorescence of the total cell population.

FIGS. 11A-11C show that the concentrations of DGP and diphyllin needed to inhibit ZIKV infection were not toxic to HT1080 (FIG. 11A), CHME3 (FIG. 11B), or VERO cells (FIG. 11C).

FIG. 12 shows ability of DGP to block infection in CHME3 cells was tested in four other ZIKV strains: PRVABC59 (Puerto Rico), DAK ArD-51254 (Senegal), IbH30656 (Nigeria), and the strain recently implicated in the 2016 outbreak, iBeH819015 (Brazilian).

FIGS. 13A-B shows the effect of DGP on viral replication in the brain (FIG. 13A) and spleen (FIB. 13B) was investigated in ZIKV-challenged mice.

FIGS. 14A-B. Human cells A549 expressing ACE2, analyzed by Western blot, (FIG. 14A). Cells expressing ACE2 were challenged with SARS Corona Virus 2 expressing GFP as a reporter (SARS-CoV-2-GFP) for 72 hours in the presence of the indicated concentrations of DGP (FIG. 14 B). Subsequently, infection was measured by determining the percentage of GFP-positive cells using flow cytometry. As shown, 0.25 μM DGP is sufficient to potently block the entry of SARS-CoV-2-GFP. These results demonstrated that DGP potently inhibits SARS-CoV-2.

### DETAILED DESCRIPTION

The inventors have discovered and demonstrated that a natural product, 6-deoxyglucose-diphyllin (DGP), also known as Patentiflorin A, and referred to herein as either DGP or Formula I, prevents and treats flavivirus infection, filovirus infection, SARS-CoV-1, SARS-CoV-2 (COVID-19) infection, and MERS-CoV infection in human cells. Diphyllin referred to herein as Formula II, prevents and treats flavivirus infection, SARS-CoV-1 infection, SARS-CoV-2 (COVID-19) infection, and a MERS-CoV infection. This was a surprising and unexpected result because DGP was originally identified as a topoisomerase Il α inhibitor, with potential anti-cancer properties. More recently DGP has been shown to inhibit certain human immunodeficiency virus-1 (HIV-1) strains.

The disclosure shows that DGP and diphyllin exhibit anti-ZIKV activity both in vitro and in vivo. DGP potently blocks ZIKV infection across all human and monkey cell lines tested. DGP also displays broad-spectrum antiviral activity against other flaviviruses. Remarkably, DGP prevents ZIKV-induced mortality in mice lacking the type I interferon receptor (Ifnar1^{-/-}). Cellular and virological experiments showed that DGP blocks ZIKV at a pre-fusion step or during fusion, which prevented the delivery of viral contents into the cytosol of the target cell. Mechanistic studies reveal that DGP and diphyllin prevent the acidification of endosomal/lysosomal compartments in target cells, thus inhibiting ZIKV fusion with cellular membranes and preventing or inhibiting infection.

The disclosure also shows that DGP (Formula I) potently blocks entry of SARS-CoV-2 in cells expressing ACE2.

### Chemical Description and Terminology

Compounds are described using standard nomenclature. Unless defined otherwise, all technical and scientific terms used herein have the same meaning as is commonly understood by one of skill in the art to which this invention belongs. Unless clearly contraindicated by the context each compound name includes the free acid or free base form of the compound as well as all pharmaceutically acceptable salts of the compound.

The term “compounds of Formula I” encompasses all compounds that satisfy Formula I, including any enantiomers, racemates and stereoisomers, as well as all pharmaceutically acceptable salts of such compounds. The term “compounds of Formula II” encompasses all compounds that satisfy Formula II, including any enantiomers, racemates and stereoisomers, as well as all pharmaceutically acceptable salts of such compounds. A dash (“-”) that is not between two letters or symbols is used to indicate a point of attachment for a substituent.

The terms “a” and “an” do not denote a limitation of quantity, but rather denote the presence of at least one of the referenced item. The term “or” means “and/or”. The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

“Pharmaceutical compositions” are compositions comprising at least one active agent, such as a compound or salt of Formula I and or Formula II, and at least one other substance, such as an excipient. An excipient can be a carrier, filler, diluent, bulking agent or other inactive or inert ingredients. Pharmaceutical compositions optionally contain one or more additional active agents. When specified, pharmaceutical compositions meet the U.S. FDA's GMP (good manufacturing practice) standards for human or non-human drugs.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

“Treating,” as used herein includes providing a compound of this disclosure such as a compound or salt of Formula I or II, either as the only active agent or together with at least one additional active agent sufficient to: (a) inhibit the disease, i.e. arrest its development; and (b) relieve the disease, i.e., causing regression of the disease and in the case of a bacterial infection to eliminate or reduce the virulence of the infection in the subject.

“Preventing” means administering an amount of a compound of the disclosure sufficient to significantly reduce the likelihood of a disease from occurring in a subject who may be predisposed to the disease but who does not have it. In the context of viral infection “preventing” includes administering an amount of a compound of Formula I or Formula II or salt thereof to a subject known to be at enhanced risk of viral infection, such as a health care worker likely to be in contact with infected individuals, a family member of an infected individual, or a person living in or traveling in an area where carriers of the infections, such as mosquito or tick carriers of the viral infection, are common. For example, prophylactic treatment may be administered when a subject is known to be at enhanced risk of viral respiratory infection, such cystic fibrosis or ventilator patients.

A “therapeutically effective amount” of a pharmaceutical composition/combination is an amount effective, when administered to a subject, to provide a therapeutic benefit, such as to decrease the morbidity and mortality associated with viral infection and/or effect a cure. In certain circumstances a subject suffering from a viral infection may not present symptoms of being infected. Thus a therapeutically effective amount of a compound is also an amount sufficient to significantly reduce the detectable level of virus in the subject's blood, serum, other bodily fluids, or tissues. In the context of prophylactic or preventative treatment, a “therapeutically effective amount” is an amount sufficient to significantly decrease the incidence of contracting the viral infection associated with viral exposure.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

An “active agent” is a compound or biological molecule, such as a naturally occurring or non-naturally occurring protein, peptide, hormone, or antibody that exhibits biological activity, such as inhibiting bacteria growth or reproduction, or potentiates the biological activity of a compound of Formula I or Formula II.

A significant reduction is any detectable negative change that is statistically significant in a standard parametric test of statistical significance such as Student's T-test, where p<0.05.

### Chemical Description

The disclosure provides compounds and salts of Formula I and Formula II. The terms “Formula I” and “Formula II” include the pharmaceutically acceptable salts of Formula I and/or II unless the context clearly indicates otherwise. In certain situations, the compounds of Formula I and/or Formula II may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms, with all isomeric forms of the compounds being included in the present disclosure. In these situations, single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example using a chiral HPLC column.

Where a compound exists in various tautomeric forms, the invention is not limited to any one of the specific tautomers, but rather includes all tautomeric forms.

The present disclosure includes all isotopes of atoms occurring in the present compounds. Isotopes include those atoms having the same atomic number but different mass numbers. By way of general example, and without limitation, isotopes of hydrogen include tritium and deuterium and isotopes of carbon include ¹⁰C, ¹³C, and ¹⁴C.

The inventors hereof discovered that DGP inhibits ZIKV infection in monkey and human cell lines (in vitro) and in mice (in vivo). In addition, DGP showed broad-spectrum antiviral activity by blocking other flaviviruses such as DENV1, TBEV, WNV, JEV and filoviruses such as Ebolavirus (EBV). Mechanistic studies revealed that DGP inhibits ZIKV infection at a pre-fusion step or during fusion of the virus. The inventors found that DGP prevents the acidification of endosomes and therefore, inhibits the fusion of the viral membrane with the cellular membrane.

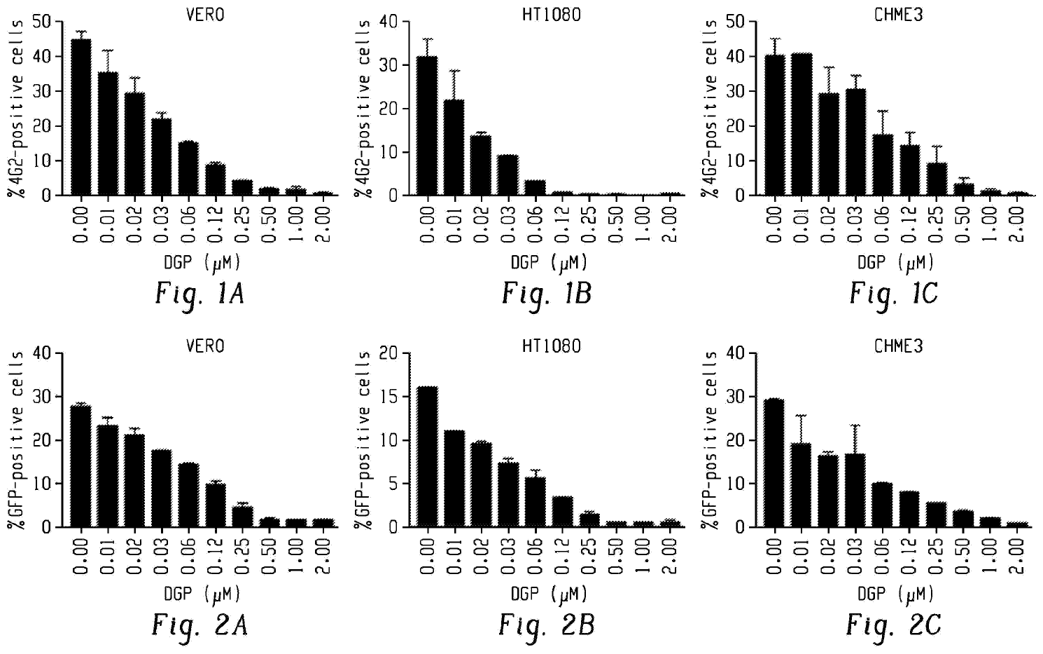

The ability of DGP to block ZIKV infection was tested in three different cell lines: African green monkey kidney epithelial cells (VERO), human fibroblast cells (HT1080), and human microglial cells (CHME3). Cells were challenged with the ZIKV strain MR766 (FIGS. 1A-1C) at a multiplicity of infection (MOI) of 1 for 48 hours in the presence DGP at the indicated concentrations. ZIKV infection was measured based on the expression of the ZIKV envelope in infected cells, which was detected via flow cytometry in fixed/permeabilized cells using the antibody 4G2. DGP potently blocked infection of the ZIKV strain MR766 in different cell lines in a dose-dependent manner (FIG. 1A-1C). A substantial inhibition of ZIKV infection when using 0.25-0.50 μM of DGP was observed, thus revealing the potency of DGP in vitro.

To corroborate these findings, the ability of DGP to block ZIKV infection by using a ZIKV-reporter virus (ZIKV-RVP) that expressed green fluorescent protein (GFP) was investigated. VERO, HT1080, and CHME3 cells each were challenged with ZIKV-RVP at an MOI of 0.5 for 48 hours in the presence of DGP at the indicated concentrations (FIGS. 2A-2C). ZIKV-RVP infection was measured by detecting GFP expression using flow cytometry. ZIKV infection was substantially inhibited at concentrations of 0.25-0.50 μM of DGP in all three cell types, regardless of the species.

To show that the ZIKV inhibitory concentrations of DGP were not toxic to cells, the 3-(4,5-dimethylthiazol-2-yl)-2,5-diphenyltetrazolium bromide (MTT) assay, which measures conversion of MTT to its insoluble form formazan was used. Overall, these results (not shown) demonstrated that DGP is a potent and non-toxic inhibitor of ZIKV infection in human and primate cell lines.

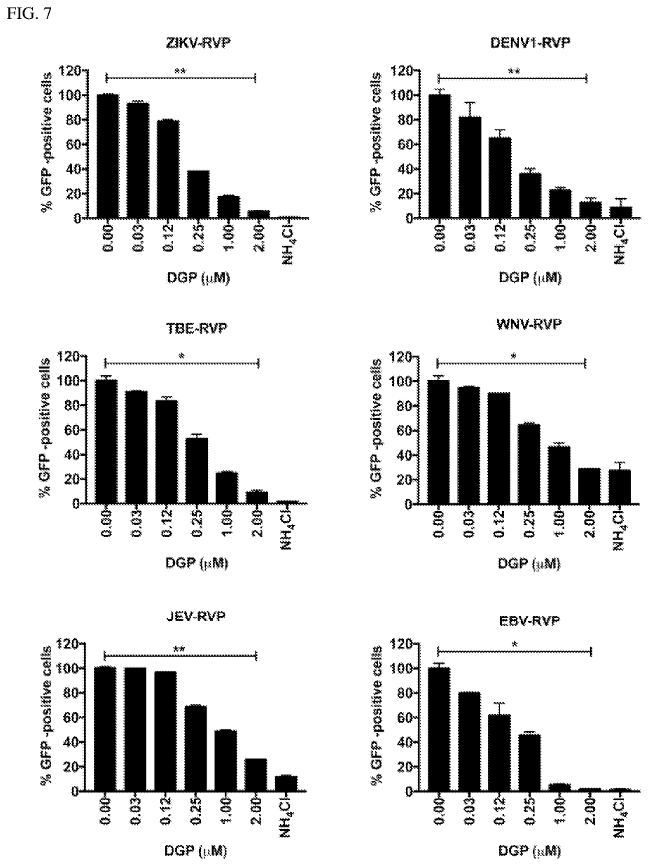

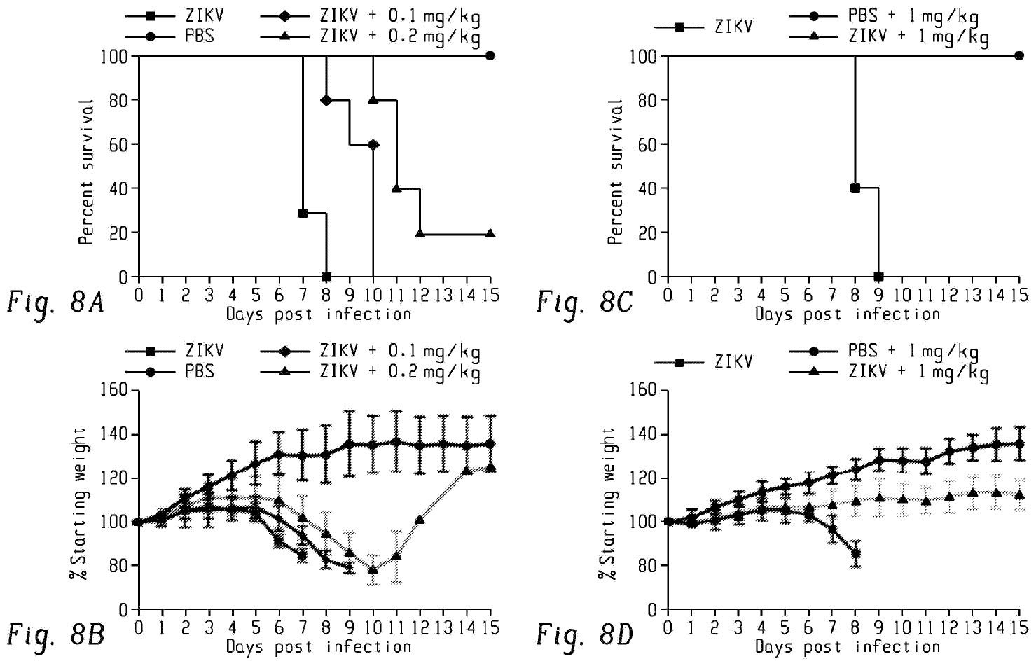

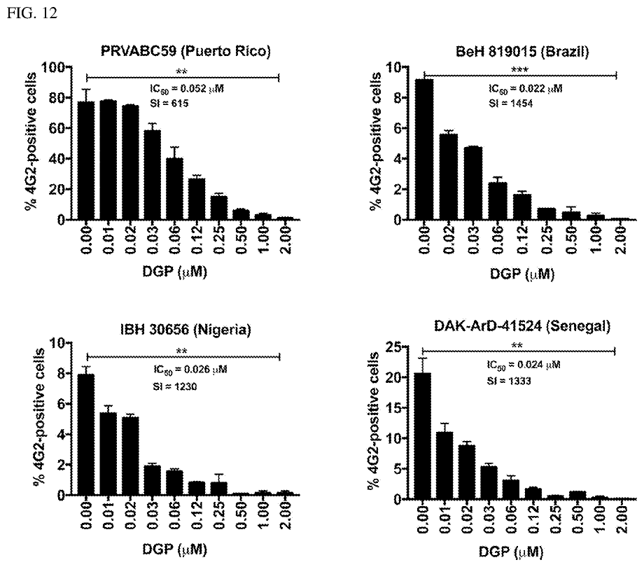

The ability of DGP to block infection in CHME3 cells was tested in four other ZIKV strains: PRVABC59 (Puerto Rico), DAK ArD-51254 (Senegal), IbH30656 (Nigeria), and the strain recently implicated in the 2016 outbreak, iBeH819015 (Brazilian) (see FIG. 12) as well as one filovirus strain (Ebola virus, EBV, data not shown). For this purpose, reporter viral particles (RVPs) expressing GFP were used as a reporter of infection, and containing the envelope of: DENV1, TBEV, WNV, JEV, or EBV. As control, cells were infected with the RVPs in the presence of 20 mM of NH₄Cl, which inhibited infection of all the tested RVPs (FIG. 7). DGP showed dose-dependent inhibitory activity against all the RVPs tested and the infection was almost undetectable at 1 μM (FIG. 7). DGP-mediated inhibition of RVP-infection was comparable to that mediated by NH₄Cl. These results demonstrate that DGP exerts antiviral activity against different flaviviruses and a filovirus, showing its potential use as a broad-spectrum antiviral agent.

To determine the ZIKV life cycle stage at which DGP acts, two approaches were used to measure the production of viral RNA: 1) In situ hybridization to image viral RNA by using fluorescent probes, and 2) Reverse transcription PCR (qRT-PCR) to quantify viral RNA. To image the viral RNA, VERO cells were challenged with the ZIKV strain MR766 at an MOI of 0.5 in the presence of 1 μM DGP, which potently blocked infection. At 48 hours post-infection, cells were fixed/permeabilized and ZIKV positive-strand RNA and detected by in situ hybridization using a specific fluorescently labeled negative-strand probe (green) and cell nuclei were stained using 4%6-diamidino-2-phenylindole (DAPI; blue). Twenty-five random images were captured for each treatment (Mock, ZIKV MR766, and ZIKV MR766+DGP 1 μM). To quantify the extent of infection, 400 cells per treatment were randomly counted and the percentage of infected (green) cells were calculated. The results demonstrated that viral RNA was substantially reduced in the presence of DGP suggesting that DGP blocks ZIKV infection before or during viral RNA synthesis (Table 1).

TABLE 1
Results from in situ hybridization.

| | Cells | Infected Cells | |
|---|---|---|---|
| | (Blue) | (Green) | % Infected Cells |
| Mock | 400 | 0 | 0 |
| ZIKV MR766 | 400 | 153 | 38.27 |
| ZIKV MR766 + DGP | 400 | 12 | 3.05 |
| (1 μM) | | | |

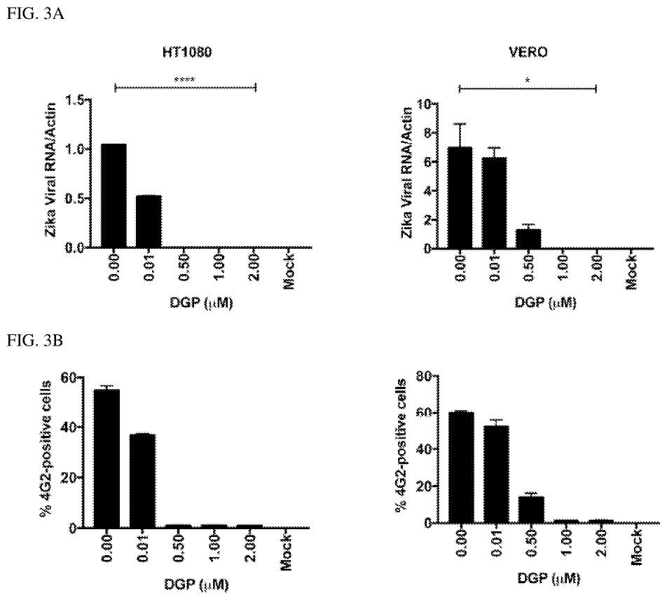

To corroborate these findings, quantitative real-time PCR (qRT-PCR) was used to quantify viral RNA using specific primers for the ZIKV genome (FIG. 3A). VERO and HT1080 cells were challenged with ZIKV at an MOI of 1 in the presence of DGP at the indicated concentrations. At 48 hours post-infection, ZIKV RNA was quantified by qRT-PCR, and normalized to Actin. The synthesis of viral RNA was completely inhibited in the presence of increasing concentrations of DGP in both cell lines. Inhibition of viral RNA production correlated with the inhibition of viral infection (compare FIG. 3A with FIG. 3B). These results suggest that DGP blocks ZIKV infection before or during viral RNA synthesis.

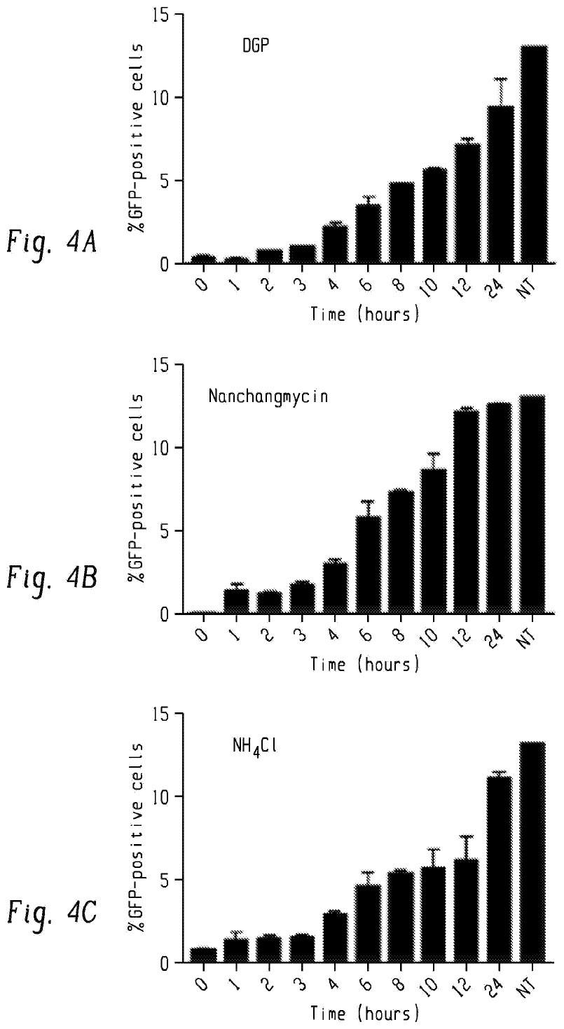

To investigate whether DGP imposes a pre- or post-fusion block to ZIKV infection, time-of-drug-addition experiments were performed for DGP and the pattern of inhibition was compared to that of known pre-fusion inhibitors of ZIKV infection, Nanchangmycin and NH₄Cl (FIGS. 4A-4C). HT1080 cells were challenged with ZIKV-RVP at an MOI of 0.5, and 1 μM of DGP (FIG. 4A), 1 μM of Nanchangmycin (FIG. 4B), or 20 mM of NH₄Cl (FIG. 4C) were added at the indicated time points. Infection was measured at 48 hours post-infection by calculating the percentage of GFP-positive cells. The inhibition of infection was stronger when the drug was added at earlier time points for DGP, Nanchangmycin, and NH₄Cl, suggesting that DGP imposes a pre-fusion block to ZIKV infection.

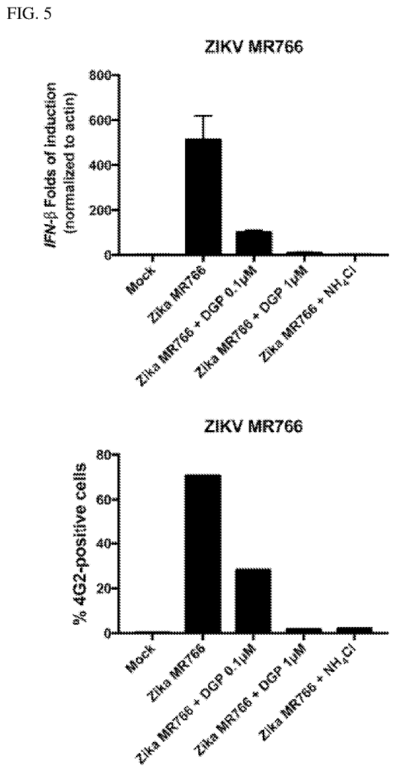

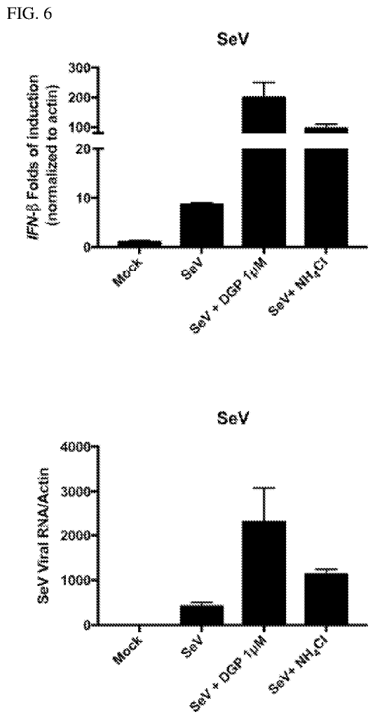

ZIKV infection activates the type I IFN response via IFN-stimulated genes that are activated by the host after recognition of viral components. If ZIKV is inhibited at a pre-fusion step, viral nucleic acids and proteins will not be exposed to the host cytosol; thus the type I IFN response will not be activated. To test whether DGP treatment prevents activation of the type I IFN response, CHME3 cells we challenged using ZIKV MR766 at an MOI of 1 in the presence of different DGP concentrations. At 48 hours post-challenge, the type I IFN response was assessed using qRT-PCR to measure IFN-β induction (FIG. 5). Treatment with DGP at both 0.1 μM and 1 μM substantially decreased and/or prevented the activation of the type I IFN response (FIG. 5, upper panel). In both DGP and NH₄Cl treatments, viral infection was inhibited, as demonstrated by the substantial decrease in the percentage of 4G2-positive cells (FIG. 5, lower panel). These results support that DGP inhibits ZIKV infection prior to or during the fusion step.

The Flaviviridae family of viruses encode a glycoprotein that is necessary to achieve fusion at the endosomal/lysosomal membranes, the step that releases viral components into the cytoplasm. To further understand the mechanism of DGP action, the effects of DGP were investigated on Sendai Virus (SeV), a virus that does not require the fusion step at the endosomal membrane to complete its replication cycle. Instead, SeV fuses at the plasma membrane. To this end, IFN-β production and viral infection were measured in CHME3 cells infected with SeV at an MOI 1 and 10 in the presence of DGP (1 μM) or 20 mM NH₄Cl. DGP did not inhibit IFN-β production in SeV-infected cells (FIG. 7, upper panel) nor di DGP inhibit SeV infection (FIG. 7, lower panel). Interestingly, treatment of SeV-infected CHME3 cells with each of DGP and NH₄Cl resulted in an increased induction of IFN-β and increased viral replication. These results indicate that DGP does not inhibit SeV infection or the resultant induction of the type I IFN response.

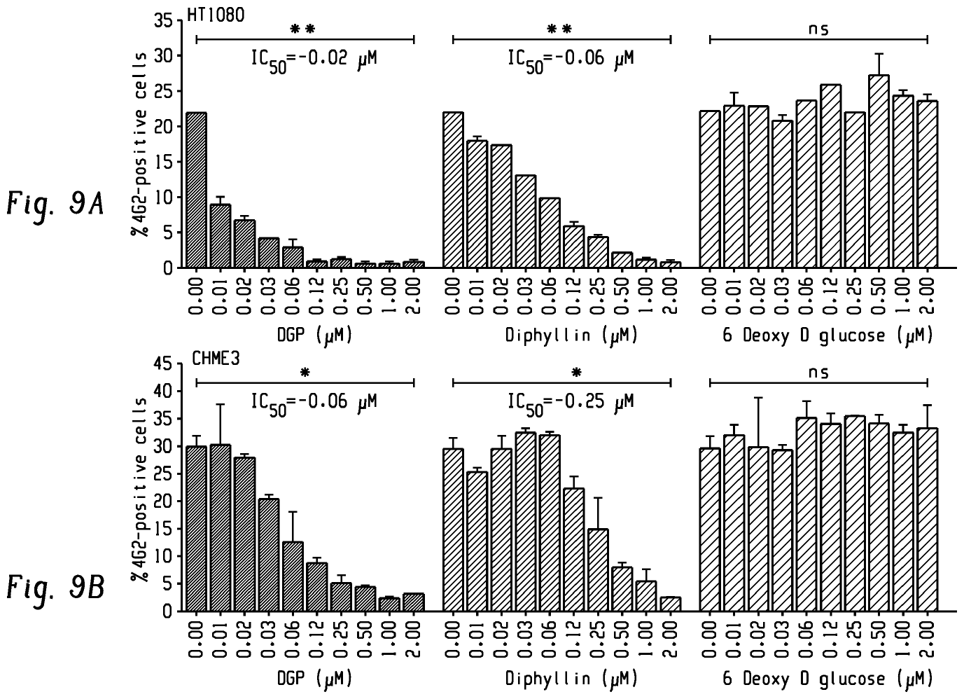

The antiviral activity of DGP in vivo was assessed by using the mouse model C57BL/6 Ifnar1^{-/-} which is a knockout mouse for the type I IFN receptor a and (3. To this end, the footpads of Ifnar1^{-/-} mice were subcutaneously inoculated using 5 plaque forming units (PFUs) of the ZIKV strain MR766, which provides a lethal dose of virus. Mice were divided in groups (6 mice/group) and injected with the following: phosphate-buffered saline (PBS; Mock-infected); ZIKV+0.1 mg/kg of DGP; or ZIKV+0.2 mg/kg of DGP (FIGS. 9A-9B) Body weight and virus-induced symptoms were monitored daily in the mice for 15 post-challenge days. The group inoculated with ZIKV showed a rapid decrease in body weight, and succumbed to viral infection at 7-8 days post-challenge, as shown by the Kaplan-Meier plot (FIG. 9A). This group displayed the following phenotypes: limb paralysis, lethargic behavior, tremors, and weight loss. The group that was challenged with ZIKV+0.1 mg/kg of DGP showed similar symptoms and succumbed to viral infection at 10 days post-challenge (FIG. 9A). However, the group injected with ZIKV+0.2 mg/kg of DGP showed a delay in the appearance of symptoms when compared with the ZIKV group, and some mice survived until day 14. These results indicate that DGP delayed the appearance of symptoms and delayed ZIKV-induced mortality compared with control mice.

To test whether increasing DGP concentrations increased survival in ZIKV-infected mice, three groups (6 mice/group) of mice were injected with the following: ZIKV; PBS+1 mg/kg DGP; or ZIKV+1 mg/kg DGP (FIG. 8C-8D). The weight and virus-induced symptoms were monitored daily in the mice for 15 post-challenge days. As previously observed, the group inoculated with ZIKV showed a rapid decrease in body weight, and succumbed to viral infection 7-9 days post-challenge, as shown by the Kaplan-Meier plot. In contrast, all six mice in the group challenged with ZIKV+1 mg/kg of DGP survived for the length of the experiment (FIG. 8C). Although in the group injected with ZIKV+1 mg/kg of DGP had lower body weights when compared to the group that was injected PBS+1 mg/kg of DGP (FIG. 8D), the group injected with ZIKV+1 mg/kg of DGP did not show any obvious disease symptoms during the course of both experiments. These results demonstrated that DGP effectively inhibits ZIKV infection in vivo when co-injected with ZIKV, thus suggesting that DGP could potentially be used as a prophylactic measure or for the treatment of ZIKV infection.

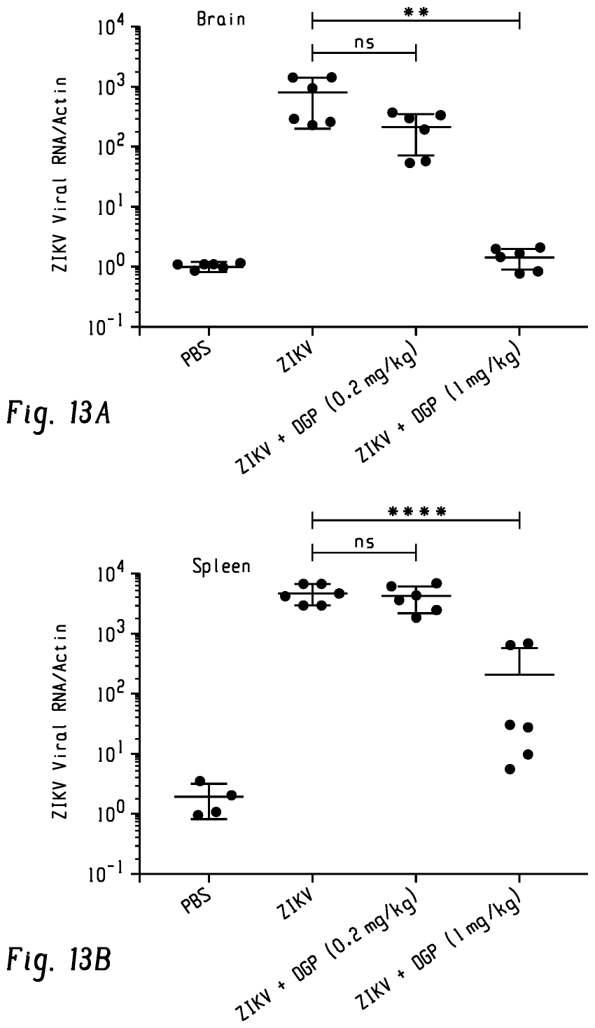

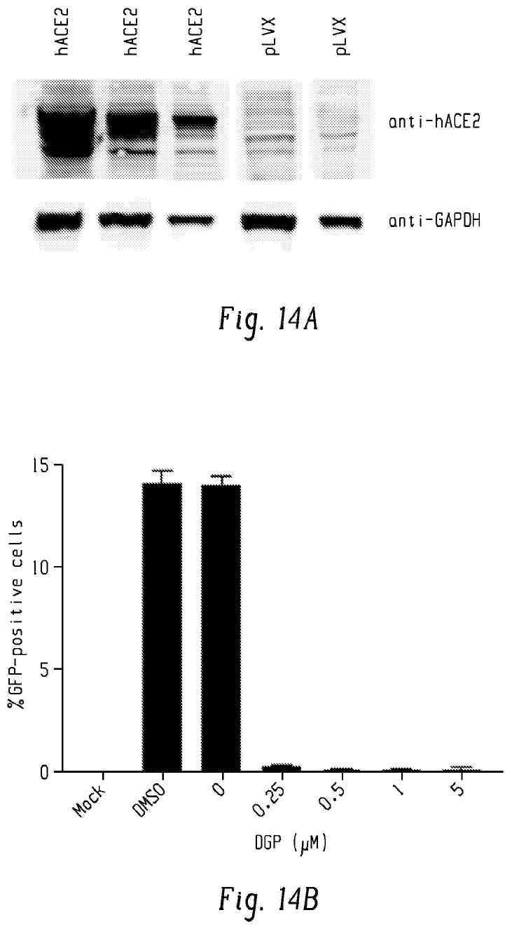

The effect of DGP on viral replication in the brain and spleen was investigated in ZIKV-challenged mice (FIG. 13). To this end, viral loads were determined by qRT-PCR using specific primers against the ZIKV genome six days post-challenge. Administration of 1 mg/kg of DGP completely inhibited viral replication in the brain (data not shown), which correlates with protection against ZIKV-induced death. However, low levels of viral replication were detected in the spleen, which may not be sufficient to cause death. These experiments suggested that DGP prevents viral replication in the brain, hence potentially conferring a higher rate of survival.

DGP prevents ZIKV-induced mortality in the type I Interferon receptor knockout mice, thus demonstrating the potential of DGP to inhibit ZIKV infection in vivo. Only a few compounds have been described to protect from ZIKV infection in vivo: chloroquine (50-100 mg/kg in mice) and the related hydroxychloroquine. These drugs has been studied for its ability to inhibit mother-to-child transmission of ZIKV in mice. Chloroquine and hydroxychloroquine are Food and Drug administration (FDA)-approved drugs to treat malaria, and they can also be used to treat ZIKV infections. However, the required inhibitory concentrations of these drugs for ZIKV in cell culture are in the micromolar range. Although more extensive testing of DGP in vivo is required, the present results demonstrate that DGP may be effective at lower doses than chloroquine and/or hydroxychloroquine to inhibit viral infection in vivo.

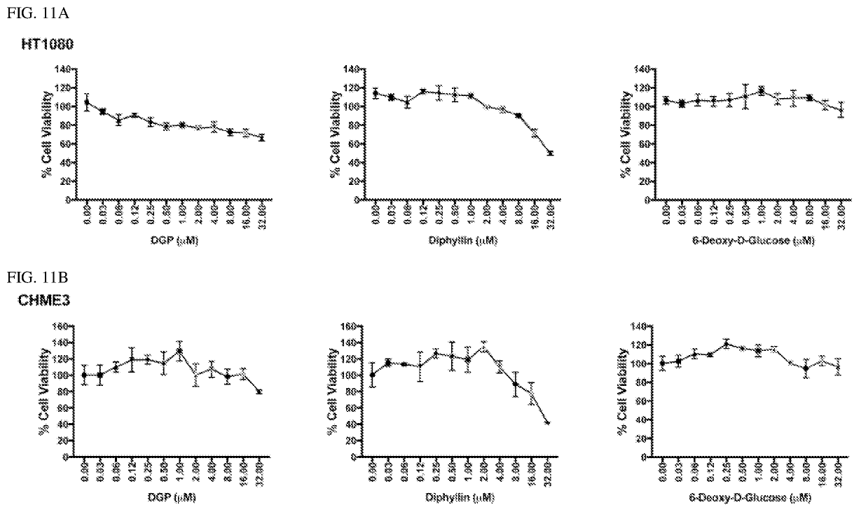

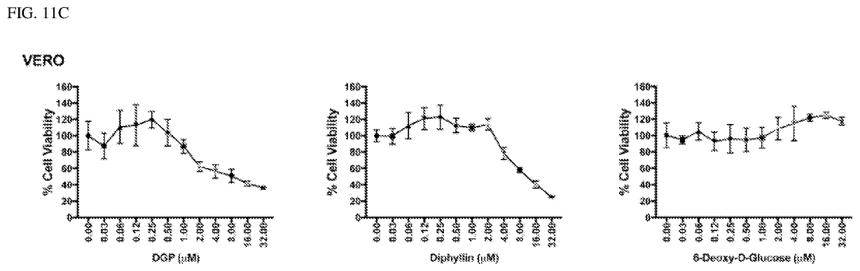

To investigate the importance of the 6-deoxyglucose (6DG) group to the biological activity of DGP, the inhibitory activities of DGP, diphyllin, and 6DG were assessed against ZIKV-infected HT1080 and ZIKV-infected CHME3 cells. Diphyllin blocked ZIKV infection in HT1080 cells with a half maximal inhibitory concentration (IC₅₀) of 0.06 μM, whereas the IC₅O of DGP was 0.02 μM (FIG. 9A). Similarly, for CHME3 cells (FIG. 9B), the IC₅₀ for diphyllin was 0.21 μM, whereas the IC₅₀ of DGP was 0.04 μM. Interestingly, DGP was 3-5-fold more potent than diphyllin, which indicates that the 6DG group contributes to increased antiviral activity. As control, the MTT assay was used to show that the concentrations of diphyllin and DGP required to inhibit ZIKV infection were not toxic to human or monkey cells (FIGS. 11A-11C).

Previous studies have shown that diphyllin affects the expression of vacuolar-ATPase, resulting in changes to the pH gradients in cells. Vacuolar-ATPases are cellular proton pumps that are crucial for processes that maintain pH gradients in the cell, such as the acidification of endosomes. Further, it has been previously suggested in the literature that ZIKV infection is affected by inhibiting endosomal acidification.

To determine whether DGP inhibited ZIKV infection by preventing the acidification of endosomes and lysosomes, Acridine Orange (AO) was used, a cell-permeable fluorescent dye marker that accumulates in low pH compartments such as endosomes and lysosomes. Within these acidic cellular compartments, AO displays orange fluorescence; however, this orange fluorescence dramatically decreases in the presence of compounds that prevent acidification of endosomes, such as the vacuolar ATPase inhibitor Bafilomycin A1.

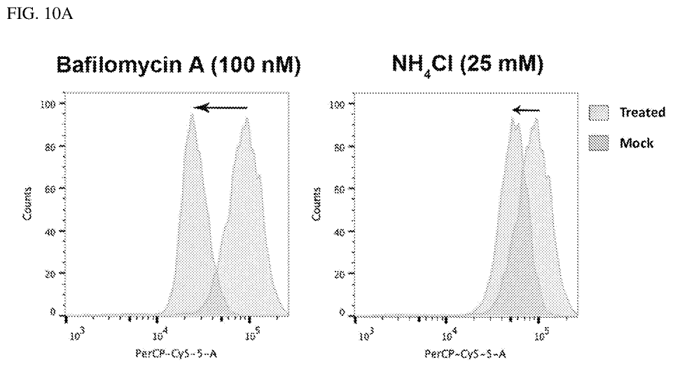

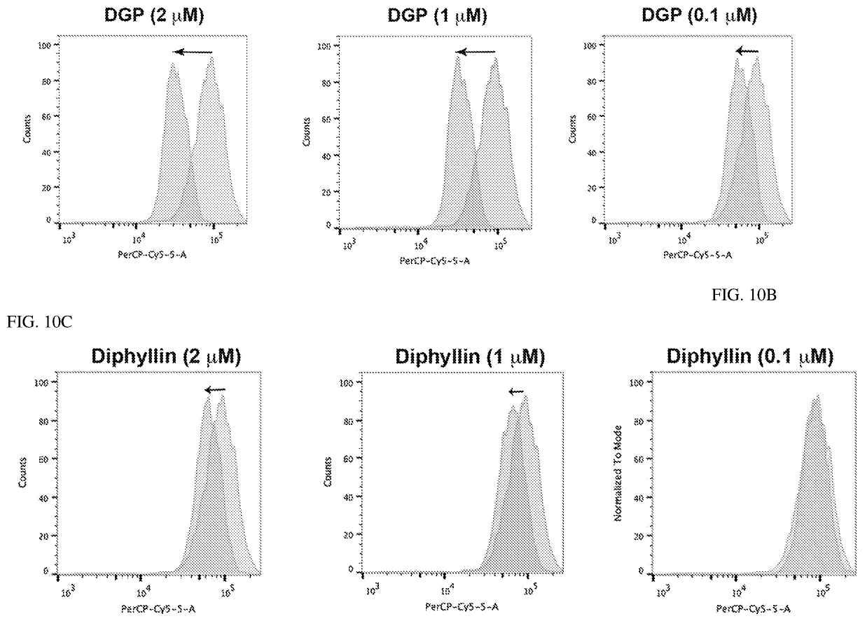

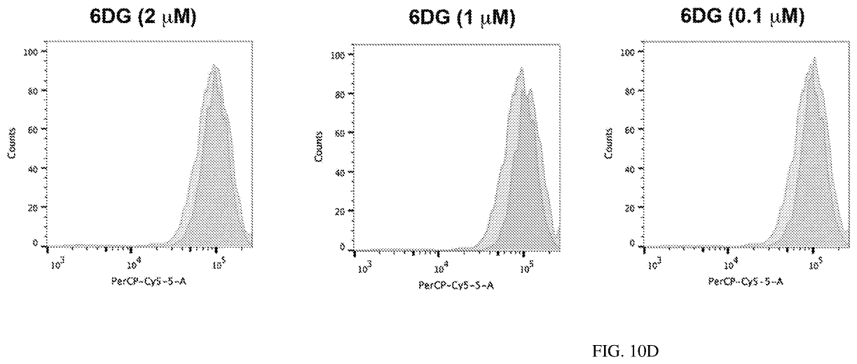

HT1080 cells were pre-incubated for 4 hours with the Bafilomycin (FIG. 10A, 100 nM), NH₄Cl (FIG. 10A, 25 mM), DGP (FIG. 10B: 2 μM, 1 μM, or 0.1 μM) diphyllin (FIG. 10C: 2 μM, 1 μM, or 0.1 μM), or 6DG (FIG. 10C: 2 μM, 1 μM, or 0.1 μM) to test whether DGP prevents endosomal/lysosomal acidification. After staining the cells with 1 μg/mL of AO, changes in fluorescence were measured using a Celesta flow cytometer on the PerCP-Cy5-5-A channel. A shift in the AO fluorescence is indicated with an arrow in FIGS. 10A-10D. As shown in FIG. 10B, increasing DGP concentrations resulted in decreased AO fluorescence in HT1080 cells, suggesting that DGP prevents the acidification of the endosomal and lysosomal compartments. As positive controls, Bafilomycin Al and NH₄Cl were used (FIG. 10A), both of which prevent endosomal and lysosomal acidification. Comparing FIG. 10A and FIG. 10B, Bafilomycin Al and DGP showed similar decreases in AO fluorescence. Diphyllin also prevented the acidification of endosomes and lysosomes but to a lesser extent when compared with DGP (FIG. 10C). 6DG did not prevent the acidification of endosomes and lysosomes (FIG. 10D). These results suggest that DGP prevents the acidification of endosomes/lysosomes, which is required for the fusion of ZIKV, thus resulting in the inhibition of ZIKV infection.

### Pharmaceutical Preparations

Compounds disclosed herein can be administered as the neat chemical, but are preferably administered as a pharmaceutical composition. Accordingly, the disclosure provides pharmaceutical compositions comprising a compound or pharmaceutically acceptable salt of Formula I and/or Formula II, together with at least one pharmaceutically acceptable carrier. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Excipients include carriers, diluents, and other inactive ingredients and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of excipients include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present disclosure.

The pharmaceutical compositions/combinations can be formulated for oral, parenteral, or intravenous administration. These compositions contain between 0.1 and 99 weight % (wt. %) of a compound of Formula I and usually at least about 5 wt. % of a compound of Formula I. Some embodiments contain from about 25 wt. % to about 50 wt. % or from about 5 wt. % to about 75 wt. % of the compound of Formula.

### Methods of Treatment

The disclosure provides methods treating or preventing a flavivirus infection, a filovirus infection, a SARS-CoV-1 infection, a SARS Co-V-2 infection, or a MERS-CoV infection, in a subject in need thereof comprising administering to the subject a compound of Formula I in an effective amount.

The disclosure provides a method for treating or preventing a SARS-CoV-1 infection, a SARS-CoV-2 infection, or a MERS-CoV infection in a subject comprising administering a therapeutically effective amount of a compound of Formula I, Formula II, or a pharmaceutically acceptable salt of either of Formula I or II to the subject.

The disclosure provides a method of preventing or reducing an effect of flavivirus infection, filovirus infection, SARS-CoV-1 infection, SARS-CoV-2 infection, or MERS-CoV infection, comprising administering a therapeutically effective amount of compound of Formula I.

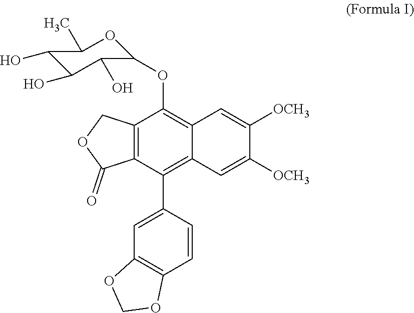 [SMILES: COc1cc2c(OC3OC(C)C(O)C(O)C3O)c3c(c(-c4ccc5c(c4)OCO5)c2cc1OC)C(=O)OC3]

or a pharmaceutically acceptable salt thereof, to a patient in need thereof, wherein the effect is inhibiting the synthesis of viral RNA, preventing the acidification of endosomes, preventing the acidification of lysosomes, inhibiting infection prior to membrane fusion, or a combination of any of the foregoing.

The disclosure provides a method of preventing or reducing an effect of a flavivirus infection, a SARS-CoV-1 infection, a SARS-CoV-2 infection, or a MERS-CoV infection, comprising administering a therapeutically effective amount of compound of Formula II

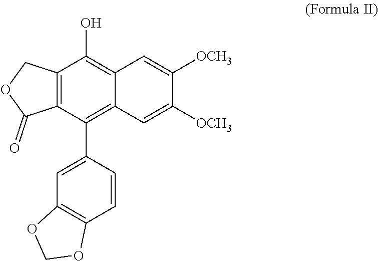 [SMILES: COc1cc2c(O)c3c(c(-c4ccc5c(c4)OCO5)c2cc1OC)C(=O)OC3]

or a pharmaceutically acceptable salt thereof, to a patient in need thereof, wherein the effect is inhibiting the synthesis of viral RNA, preventing the acidification of endosomes, preventing the acidification of lysosomes, inhibiting infection prior to membrane fusion, or a combination of any of the foregoing.

The disclosure also provides methods treating or preventing a flavivirus infection in a subject comprising administering to the subject an effective amount of a compound of Formula IIa compound of Formula II in an effective amount.

Compounds of Formula I and Formula II can treat or prevent a flavivirus infection in a subject. The subject can have, or be exposed to, for example, a virus from the Flaviviridae family of viruses. Members of this family belong to a single genus, flavivirus, and cause widespread morbidity and mortality throughout the world. Mosquito-transmitted flaviviruses include: Yellow Fever, Dengue Fever, Japanese encephalitis, West Nile viruses, and Zika virus. Flaviviruses transmitted by ticks include Tick-borne Encephalitis (TBE), Kyasanur Forest Disease (KFD) and Alkhurma disease, and Omsk hemorrhagic fever.

Compounds of Formula I can treat or prevent a Filovirus infection in a subject. The subject can have or be exposed to, for example, a virus from the Filoviridae family of viruses (Cuevavirus, Marburgvirus and Ebolavirus) which can cause severe hemorrhagic fever in humans and nonhuman primates. Ebolavirus includes the following: Ebola virus (species Zaire ebolavirus), Sudan virus (species Sudan ebolavirus), TaI Forest virus (species Taï Forest ebolavirus, formerly known as Côte d'lvoire ebolavirus), Bundibugyo virus (species Bundibugyo ebolavirus), Reston virus (species Reston ebolavirus), and Bombali virus (species Bombali ebolavirus). Ebola, Sudan, TaI Forest, and Bundibugyo viruses are known to infect people whereas Reston virus is known to cause disease in nonhuman primates and pigs, but not in people. Bombali virus was recently identified in bats, and it is unknown at this time if it causes disease in either animals or people.

The compound of Formula I or salt thereof can be administered as a pharmaceutical composition comprising the compound or salt of Formula I and a pharmaceutically acceptable excipient. The compound of Formula I or salt thereof can be administered as the only active agent or can be administered together with an additional active agent.

The compound of Formula II or salt thereof can be administered as a pharmaceutical composition comprising the compound or salt of Formula II and a pharmaceutically acceptable excipient. The compound of Formula II or salt thereof can be administered as the only active agent or can be administered together with an additional active agent. The disclosure also provides a method for inhibiting the synthesis of viral RNA, reducing or preventing the acidification of endosomes, lysosomes, or a combination thereof, and/or inhibiting infection prior to membrane fusion, in a subject in need thereof comprising administering to the subject a compound of Formula I and/or a compound of Formula II, in an amount effective to protect cells from viral infection.

The disclosure provides a method of inhibiting the synthesis of viral RNA in a cell, reducing acidification of endosomes in a cell, reducing acidification of lysosomes in a cell, inhibiting flavivirus infection in a cell prior to membrane fusion, or a combination of any of the foregoing, wherein the cell is a cell that has been contacted with a flavivirus to form a flavivirus-contacted cell said method comprising contacting the flavivirus-contacted cell with a sufficient concentration of a compound of Formula I, Formula II, a pharmaceutically acceptable salt thereof, or a combination of any of the foregoing.

The disclosure also provides a method of preventing the synthesis of viral RNA in a cell, preventing acidification of endosomes in a cell, preventing acidification of lysosomes in a cell, or preventing flavivirus infection in cell prior to membrane fusion, or a combination of any of the foregoing, said method comprising contacting the cell with sufficient concentration of a compound of Formula I, Formula II, a pharmaceutically acceptable salt thereof, or a combination of any of the foregoing prior to contacting the cell with a flavivirus.

In an embodiment the subject is a mammal. In certain embodiments the subject is a human, for example a human patient exposed to or infected with a flavivirus or Filovirus. The subject may also be a companion a non-human mammal, such as a companion animal, e.g. primates, cats and dogs, or a livestock animal.

For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g., blood, plasma, serum, cellular interstitial fluid, saliva, feces and urine) and cell and tissue samples of the above subjects will be suitable for use.

An effective amount of a pharmaceutical composition may be an amount sufficient to inhibit the progression of a disease or disorder, cause a regression of a disease or disorder, reduce symptoms of a disease or disorder, or significantly alter a level of a marker of a disease or disorder. In flavivirus infections, the virus can be found in serum or plasma, generally 2-7 days following disease onset, and the duration of this viremic phase and the viral load detected vary depending on the infecting virus. Examples of diagnostic methods used for the confirmation of EBV infection include antibody-capture enzyme-linked immunosorbent assay (ELISA), antigen-capture detection methods, serum neutralization test, RT-PCR assay, electron microscopy, and viral isolation by cell culture.

An effective amount of a compound or pharmaceutical composition described herein will also provide a sufficient concentration of a compound of Formula I and/or Formula II when administered to a subject. A sufficient concentration is a concentration of the compound of Formula I and/or Formula II in the patient's body necessary to prevent or combat a flavivirus infection for which a compound of Formula I or Formula II is effective. A sufficient concentration is a concentration of the compound of Formula I in the patient's body necessary to prevent or combat a filovirus infection for which a compound of Formula I is effective. Such an amount may be ascertained experimentally, for example by assaying blood concentration of the compound, or theoretically, by calculating bioavailability.

Methods of treatment include providing certain dosage amounts of a compound of Formula I and/or Formula II to a subject or patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 1000 mg or about 1 mg to about 500 mg of each active compound. In certain embodiments 1 mg to 1000 mg, 1 mg to 500 mg. 10 mg to 500 mg, 100 mg to 600 mg, 100 mg. to 500 mg, 25 mg to 500 mg, or 25 mg to 200 mg of a compound of Formula I or Formula II are provided daily to a patient. Frequency of dosage may also vary depending on the compound used and the particular disease treated. However, for treatment of most diseases and disorders, a dosage regimen of 4 times daily or less can be used and in certain embodiments a dosage regimen of 1 or 2 times daily is used.

The disclosure includes methods of treatment in which a compound of Formula I or Formula II or a salt thereof is administered at a dosage ranging from about 0.1 mg/kg to about 50 mg/kg body weight, about 0.1 mg/kg to about 25 mg/kg, about 0.5 mg/kg to about 25 mg/kg, about 0.1 mg/kg to about 20 mg/kg, about 0.1 mg/kg to about 10 mg/kg, about 1.0 mg/kg to about 10 mg/kg, about 0.1 mg/kg to about 5.0 mg/kg, about 1.0 mg/kg to about 5.0 mg/kg, based on the weight of the compound of Formula I or compound of Formula II. It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

This disclosure is further illustrated by the following examples, which are non-limiting.

### EXAMPLES

### Example 1

### Mouse Studies

Mice were purchased from Jackson Laboratories and bred in a specific-pathogen-free facility at Albert Einstein College of Medicine. C57BL/6 mice that are knockout for the type I IFN receptor alpha and beta [Stock No. 32045-JAXIFN-αβR-(lfnar1^{-/-}), Jackson Laboratories] were used for ZIKV challenges. Groups with 6 mice each (3-4 week-old, females and males) were subcutaneously injected (footpad) using 30 μl of PBS containing the indicated amount of DGP, with or without 5 PFUs of ZIKV. Mortality, symptoms, and body weight of each mouse was monitored for 15 post-challenge days.

### Cell Lines

VERO cells (ATCC CCL-81), HT1080 cells (ATCC CCL-121), and CHME3 cells (human microglia cells) were grown at 37° C. in 5% CO₂ in Dulbecco's modified Eagle's medium (DMEM) supplemented with 10% fetal calf serum (FCS), 100 IU/mL of penicillin, and 100 μg/mL of streptomycin. Cells were seeded in 24-well plates (50,000 cells/well) 24 h prior to infection with ZIKV at a multiplicity of infection indicated for each experiment.

### Viruses

ZIKV strain MR766 (a gift from Dr. Paul Bates), was the first described ZIKV strain that was isolated in the Zika Forest of Uganda in 1947 was produced and expanded in VERO cells. ZIKV strains IbH 30656 (Human/1968/Nigeria), PRVABC59 (Human/2015/Puerto Rico), and DAK AR 41524 (Mosquito/1984/Senegal) were initially obtain from Biodefense and Emerging Infection Research Resources Repository (BEI Resources, Manassas, Va.) and subsequently propagated in C6/36 cells. The Brazilian Zika strain BeH819015 (GenBank KU365778.1) virus was produced from a molecular clone generated in the Laboratory of Vector-Borne Viral Diseases (sequence available upon request) (Liu, S. et al. “AXL-Mediated Productive Infection of Human Endothelial Cells by Zika Virus.” Circ Res 119(11): 1183-1189). IbH 30656, PRVABC59, DAK AR 41524 and BeH819015 were a gift from Dr. Tony Wang.

All ZIKV strains were produced and expanded in VERO cells. For viral production, VERO cells were seeded in 10-cm plates at 24 h prior to ZIKV infection. Cells were infected with ZIKV at an MOI of 10 in DMEM supplemented media with 10% FCS, 100 IU/mL of penicillin, 100 μg/mL of streptomycin, and 25 mM HEPES for 3 h. An extra 5 mL of the same media was subsequently added. The cultures were maintained for 72 h at 37° C., after which the supernatant was collected and centrifuged for 10 min at 3000×g. ZIKV was stored in aliquots at −80° C. until further use. For virus titration, serial dilutions of ZIKV were used to challenge VERO cells and infection was determined by flow cytometry using the 4G2 antibody.

Zika, Dengue 1, West Nile, Japanese encephalitis, and tick-born encephalitis viral reporter particles (ZIKV-RVP, DENV1-RVP, WNV-RVP, JEV-RVP, and TBEV-RVP) were produced by co-transfection of two plasmids, the appropriate CPrME and WNV-NS-GFP, as previously shown (Persaud, M. et al. 2018, “Infection by Zika viruses requires the transmembrane protein AXL, endocytosis and low pH.” Virology 518: 301-312). The CPrME construct encodes the structural genes capsid (C), signal sequence, pro-membrane protein (PrM), and envelope protein (E) for each viral strain (ZIKV accession: KU312312, DENV1 accession: AHG06335.1, WNV accession: AAF20092.2, JEV accession: ADY69180.1, and TBEV accession: AAB53095.1). Sequences for ZIKV-RVP belong to the Suriname strain KU312312, which is the strain involved in a recent Brazilian outbreak of infection. To construct the reporter viruses the following strains were used: Hypr strain for TBEV, NY-99 strain for WNV, West Pacific-74 strain for DENV1, and SX095-01 for JEV. The genes for all the viruses were codon-optimized for mammalian cells and cloned into the LPCX vector. The WNV-NS-GFP plasmid encodes the non-structural genes of WNV and a GFP reporter. All except for the first 20 amino acids of the capsid and the last 28 amino acids of envelope of the WNV genome were replaced with GFP. To generate viral particles, HEK293T cells were co-transfected with 1 μg of the CPrME constructs and 5 μg WNV-NS-GFP using a polyethylimine transfection reagent at 1 mg/mL in serum-free DMEM. At 24 hours post-transfection, the media was replaced with fresh DMEM and cells were maintained for an additional 24 h. The suspension was centrifuged at 3000×g for 10 min to remove cellular debris, and the supernatant containing infectious viral particles was collected. Virus stocks were stored at −80° C. and were thawed at 37° C. immediately before use.

### Example 2

### Detection of Infection by ZIKV Strain MR766

The methodology used to detect ZIKV strain MR766 was previously described in (Persaud, M. et al. 2018). In detail, cells were seeded in 24-well plates and infected with ZIKV strain MR766 at the indicated MOI for 48 h. Subsequently, cells were detached using 5 mM ethylenediaminetetraacetic acid (EDTA) in phosphate buffered saline (PBS), collected by centrifugation, and fixed with 1.5% paraformaldehyde in PBS for 15 mM. The cells were then suspended in 0.1 M glycine for 10 mM to quench the paraformaldehyde, and then washed with PBS. Cells were blocked for 30 mM using 1χ Perm/Wash solution (BD Bioscience 51-2091KZ) in PBS and then incubated for 45 min in the same solution with anti-ZIKV E protein-specific monoclonal antibody 4G2, a gift of Dr. A. Brass.

As a control, an isotype-matched non-binding mouse IgG1 monoclonal antibody (Invitrogen Ms IgG1) was used at approximately the same concentration on replicate samples. Afterwards, cells were washed 3 times with 1×Perm Wash buffer and incubated with goat anti-mouse Alexa-fluor antibodies (Invitrogen, diluted 1:2000). Positive cells (ZIKV-infected) were detected using a Celesta flow cytometer (BD Biosciences). This method for quantitating infection was also used for titration of ZIKV MR766 stocks of VERO cells.

### Example 3

### Quantitative RT-PCR for the Detection of ZIKV and SeV

To detect viral copies of ZIKV and Sendai Virus (SeV) by qRT-PCR, cells were seeded in 24-well plates, with or without DGP treatment, and infected with the virus at the indicated MOI for 48 h. After the incubation period, total RNA from HT1080, VERO, and CHME3 cells was isolated and purified using Trizol (Invitrogen). For detection of ZIKV viral load in brain and spleen, 3 mice were sacrificed at 6 days post-infection and total RNA was extracted from the brain and spleen. For cDNA synthesis, 1 ng of total RNA was reverse transcribed. The reaction mixture included 1 mM of deoxyribonucleotide phosphates (dNTPs), 2 nM of the specific reverse primer of ZIKV or SeV, 1× M-MULV buffer, 10 U M-MuLV RT (BioLabs), and 2 U of RNase Inhibitor. The reaction mixture was incubated for 1 hour at 42° C. followed by 20 min at 65° C. to inactivate the enzyme. For actin detection, Oligo-dT was used to reverse transcribe total RNA.

ZIKV RNA levels were measured using real-time PCR. HT1080 and VERO cells were challenged by ZIKV MR766 at an MOI of 1 in the presence of DGP. At 48 hours post-challenge, cells were lysed and total RNA was extracted using trizol. Total RNA was used to determine the levels of ZIKV RNA by real-time PCR using specific primers against ZIKA. qRT-PCR was carried out using SYBR green in a 20-μl final volume using a MASTERCYCLER proS machine. The primers used to detect ZIKV were: SEQ ID NO. 1: 5′-TT GTCATGATACTGCTGATTGC-3′-Forward (Genome Position 941-964) and SEQ ID NO. 2: 5′-CGTCGTCGTGACCAACTCTA-3′-Reverse (Genome position 1123-1103) (AY632535.2). For the detection of SeV, the following primers were used: SEQ ID NO. 3: 5′-CAGAGGAGCACAGTCTCAGTGTTC-3′-Forward (Genome position 210-233) and SEQ ID NO. 4: 5′-TCTCTGAGAGTGCTGCTTATCTGTGT-3′-Reverse (Genome position 332-307) (M30202. Genome position 210-332) (Wagner, A. M. et al. 2003, “Detection of sendai virus and pneumonia virus of mice by use of fluorogenic nuclease reverse transcriptase polymerase chain reaction analysis.” Comp Med 53(2): 173-177). For the detection of IFN-β the following primers were used: SEQ ID NO. 5: Forward 5-ACCTCCGAAACTGAAGATCTCCTA-3′ (Genome position 644-668) and SEQ ID Na 6: Reverse 5′-TGCTGGTEGAAGAATGCTTGA-3′ (Genome position 718-697) (NM_002176.2) and for actin detection: SEQ ID NO. 7: 5′-AACACCCCAGCCATGTACGT-′3-Forward and SEQ ID NO. 8: 5′-CGGTGAGGATCTTCATGAGGTAGT′3-Reverse. ZIKA viral RNA levels were normalized to actin (upper panels). In parallel, similar infections were used to determine infectivity via flow cytometry using anti-4G2 antibodies (lower panels). Experiments were performed at least three times, and results of a representative experiment are shown.

### Example 4

### In Situ (+)-ZIKV RNA Hybridization

ZIKV RNA in cultured adherent cells was probed using the RNAscope reagents and protocol (Advanced Cell Diagnostics) (Wang, F. et al. 2012, “RNAscope: a novel in situ RNA analysis platform for formalin-fixed, paraffin-embedded tissues.” J Mol Diagn 14(1): 22-29) with some modifications as previously described (Puray-Chavez, M. 2017, “Multiplex single-cell visualization of nucleic acids and protein during HIV infection.” Nat Commun 8(1): 1882. Fixed cells on coverslips were washed twice with PBS, then incubated with 0.1% Tween-20 in PBS (PBS-T) for 10 min at room temperature (RT) and washed in PBS for 1 min. Coverslips were immobilized on glass slides, followed by protease treatment (Pretreat 3) that was diluted 1:2 in PBS and incubated in a humidified HybEZ oven at 40° C. for 15 min. The slides were washed twice with PBS for 1 min. ZIKV-specific target probe, V-ZIKA-pp-02, for the (+) RNA (Advanced Cell Diagnostics) was added to the coverslip and incubated in a humidified HybEZ oven at 40° C. for 2 h. Two consecutive wash steps in 1×wash buffer (Catalog number, 310091; Advanced Cell Diagnostics) were performed on a rocking platform at RT for 2 min in every wash step after this point, and all incubations were performed in a humidified HybEZ oven at 40° C. cDNA amplification was performed using a series of amplifiers (RNAscope; Advanced Cell Diagnostics). Amplifier hybridization 1-Fluorescent (Amp 1-FL) was added to the coverslip for 30 min, followed by Amp 2-FL hybridization for 15 min. Amp 3-FL hybridization was then added for 30 min, followed by Amp 4-FL hybridization for 15 min. Nuclei were stained with DAPI (Advanced Cell Diagnostics) for 1 minute at RT. Coverslips were washed 2 times in PBS, detached, and mounted on slides using ProLong Gold Antifade reagent (Thermo Fisher Scientific). Images were obtained using the Leica TCP SP8 inverted confocal fluorescence microscope using a 63×/1.4 oil-immersion objective. The excitation/emission bandpass wavelengths used to detect DAPI and Alexa-fluor 488 were set to 405/420-480 and 488/505-550, respectively. In order to quantify the differential drug effects on (+) ZIKV RNA, 25 images were manually acquired of each biological replicate drug treatment experiment and performed cellular analysis.

### Example 5

### Determination of Acridine Orange Fluorescence

Acridine Orange (Invitrogen) staining was performed as described previously (Kanzawa, T. et al. 2004, “Role of autophagy in temozolomide-induced cytotoxicity for malignant glioma cells.” Cell Death Differ 11(4): 448-457). Cells were stained with 1 μg/mL AO in 10% FBS DMEM for 30 min at 37° C. and then collected by trypsinization. Changes in fluorescence were measured using a Celesta flow cytometer in the PerCP-Cy5-5-A channel.

### Example 6

### Cell Viability Assay

Cell viability was determined by measuring the reduction of the tetrazolium dye MTT [3-(4,5-dimethylthiazol-2-yl)-2,5-diphenyltetrazolium bromide] to its insoluble form formazan. We treated 4×10³ cells/well in a 96-well plate with serial dilutions of the indicated drugs. Human and monkey cells were incubated with DGP, diphyllin, or 6-deoxy-D-glucose for 48 hours at 37° C. After the incubation period, 10 μl MTT solution (5 mg/mL) was added to each well for an additional 4 hours at 37° C. Finally, the media was removed and dimethyl sulfoxide was added (200 μl/well) according to the manufacturer's instructions. The optical density was measured at 570 nm using a microplate reader. Experiments were performed in triplicates and standard deviations are shown. Mock-treated cells represent 100% viability.

### Quantification and Statistical Analysis

To compare the effects of each treatment in relation to its control, all data was analyzed using the two-tailed Student's t-test. Differences were considered statistically significant at P<0.05 (*), P<0.01 (**), P<0.001 (***), or non-significant (ns).

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for preventing or treating a viral infection, wherein the infection is an infection by a flavivirus, a filovirus, a SARS-CoV-1 virus, a SARS-CoV-2 (COVID-19) virus, or a MERS-CoV virus, said method comprising administering to a subject in need thereof a therapeutically effective amount of a compound of Formula I or Formula II: 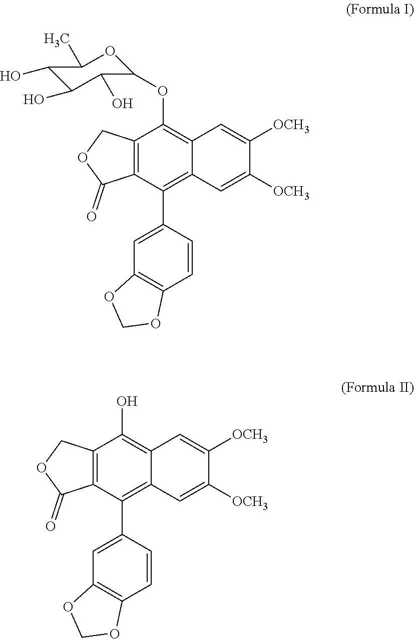 [SMILES: COc1cc2c(OC3OC(C)C(O)C(O)C3O)c3c(c(-c4ccc5c(c4)OCO5)c2cc1OC)C(=O)OC3 ; COc1cc2c(O)c3c(c(-c4ccc5c(c4)OCO5)c2cc1OC)C(=O)OC3]
or a pharmaceutically acceptable salt of a compound of Formula I or Formula II.

2. The method of claim 1, wherein the viral infection is a flavivirus infection and the flavivirus is a mosquito-transmitted flavivirus.

3. The method of claim 1, wherein the mosquito transmitted flavivirus is yellow fever, dengue Fever, Japanese encephalitis, St. Louis encephalitis, West Nile virus, or zika virus.

4. The method of claim 1, wherein the viral infection is a flavivirus infection and the flavivirus is a tick-transmitted flavivirus.

5. The method of claim 4, wherein the tick-transmitted flavivirus is Tick-borne encephalitis, Kyasanur Forest disease, Alkhurma hemorrhagic fever, or Omsk hemorrhagic fever.

6. The method of claim 1, wherein the viral infection is a filovirus infection and the filovirus is Cuevavirus, Marburgvirus, or Ebolavirus.

7. The method of claim 6, wherein the Ebolavirus is Ebola virus, Sudan virus, Taï Forest virus, Bundibugyo virus, Reston virus, Bombali virus, Sudan virus, Täi Forest virus, or Bundibugyo virus.

8. The method of claim 1, wherein the infection is a SARS-CoV-1 infection.

9. The method of claim 1, wherein the infection is a SARS-CoV-2 (COVID-19) infection.

10. The method of claim 1, wherein the infection is a MERS-CoV infection.

11. A method of preventing or reducing an effect of flavivirus infection, filovirus infection, SARS-CoV-1 infection, SARS-CoV-2 infection, or MERS-CoV infection, comprising administering a therapeutically effective amount of compound of Formula I or Formula II. 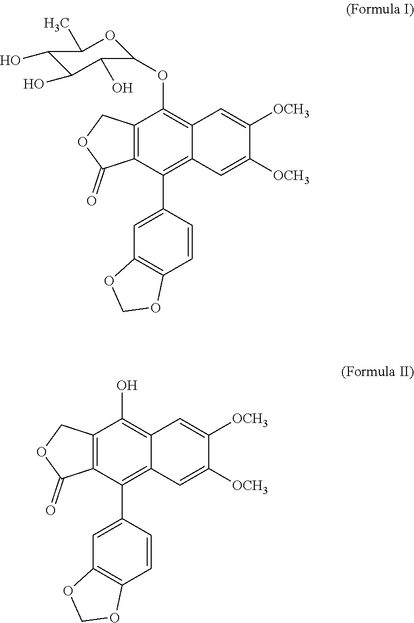 [SMILES: COc1cc2c(OC3OC(C)C(O)C(O)C3O)c3c(c(-c4ccc5c(c4)OCO5)c2cc1OC)C(=O)OC3 ; COc1cc2c(O)c3c(c(-c4ccc5c(c4)OCO5)c2cc1OC)C(=O)OC3]
or a pharmaceutically acceptable salt of Formula I or Formula II, to a patient in need thereof, wherein the effect is inhibiting the synthesis of viral RNA, preventing the acidification of endosomes, preventing the acidification of lysosomes, inhibiting infection prior to membrane fusion, or a combination of any of the foregoing.

12. The method of claim 1, wherein the compound of Formula I or Formula II or salt of Formula I or Formula II is administered as a pharmaceutical composition comprising a compound of Formula I or Formula II or salt of Formula I or Formula II, a pharmaceutically acceptable excipient, and optionally an additional active agent.

13-21. (canceled)

22. The method of claim 1, wherein the compound of Formula I or salt thereof or compound of Formula II or salt thereof is administered at a dosage ranging from about 0.1 mg/kg to about 50 mg/kg body weight based on the weight of the compound of Formula I or compound of Formula II.

23. The method of claim 1, wherein said compound or salt thereof is administered at a dosage ranging from 0.5 mg/kg to 25 mg/kg body weight based on the weight of compound.

24. The method of claim 1, wherein said compound or salt thereof is administered at a dosage ranging from 1.0 mg/kg to 5.0 mg/kg body weight based on the weight of compound.

25. The method of claim 1, wherein said compound or salt thereof is administered as a unit dose ranging from 10 mg to 500 mg based of the weight of compound.

26. A method of inhibiting the synthesis of viral RNA in a cell, reducing acidification of endosomes in a cell, reducing acidification of lysosomes in a cell, inhibiting flavivirus infection in a cell prior to membrane fusion, or a combination of any of the foregoing, wherein the cell is a cell that has been contacted with a flavivirus to form a flavivirus-contacted cell, said method comprising contacting the flavivirus-contacted cell with a sufficient concentration of a compound of Formula I, Formula II, a pharmaceutically acceptable salt thereof, or a combination of any of the foregoing, wherein Formula I and Formula II are:

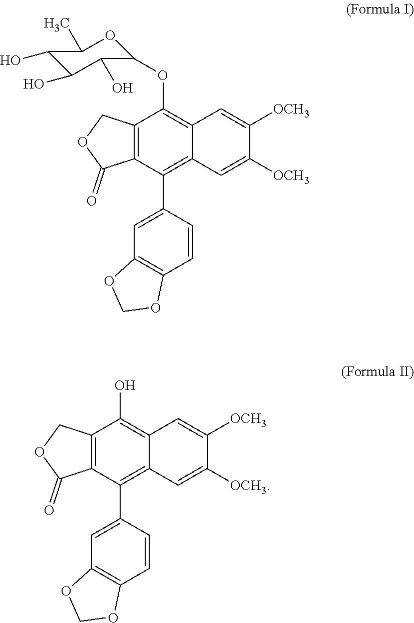 [SMILES: COc1cc2c(OC3OC(C)C(O)C(O)C3O)c3c(c(-c4ccc5c(c4)OCO5)c2cc1OC)C(=O)OC3 ; COc1cc2c(O)c3c(c(-c4ccc5c(c4)OCO5)c2cc1OC)C(=O)OC3]

27. A method of preventing the synthesis of viral RNA in a cell, preventing acidification of endosomes in a cell, preventing acidification of lysosomes in a cell, or preventing flavivirus infection in cell prior to membrane fusion, or a combination of any of the foregoing, said method comprising
contacting the cell with sufficient concentration of a compound of Formula I, Formula II, a pharmaceutically acceptable salt thereof, or a combination of any of the foregoing prior to contacting the cell with a flavivirus, wherein Formula I and Formula II are:

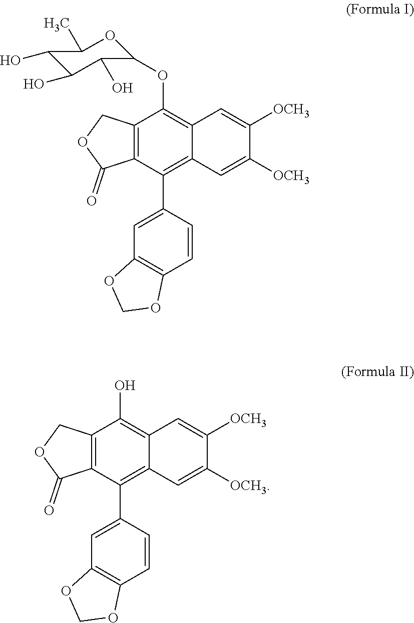 [SMILES: COc1cc2c(OC3OC(C)C(O)C(O)C3O)c3c(c(-c4ccc5c(c4)OCO5)c2cc1OC)C(=O)OC3 ; COc1cc2c(O)c3c(c(-c4ccc5c(c4)OCO5)c2cc1OC)C(=O)OC3]

28. A method of preventing or inhibiting at least one of the synthesis of viral RNA in a cell, acidification of endosomes in a cell, acidification of lysosomes in a cell, binding of a SARS-CoV-1 virus to an ACE2 receptor on a cell, binding of a SARS-CoV-2 virus to an ACE2 receptor on a cell, or binding of a MERS-CoV virus to an DPP4 receptor on a cell, said method comprising contacting the cell with sufficient concentration of a compound of Formula I, Formula II, a pharmaceutically acceptable salt thereof, or a combination of any of the foregoing prior to contacting the cell with a SARS-CoV-virus, a SARS-CoV-2 virus, or a MERS-CoV virus, wherein Formula I and Formula II are: 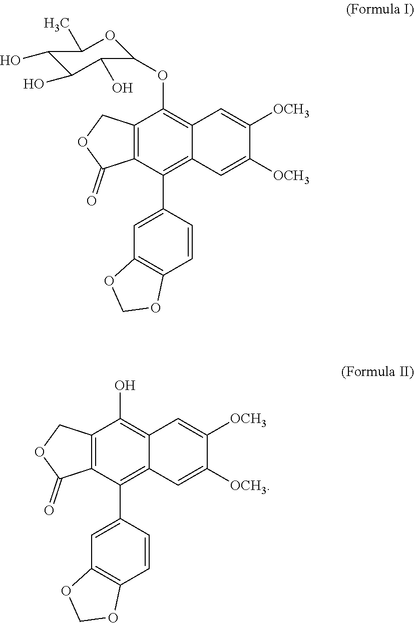 [SMILES: COc1cc2c(OC3OC(C)C(O)C(O)C3O)c3c(c(-c4ccc5c(c4)OCO5)c2cc1OC)C(=O)OC3 ; COc1cc2c(O)c3c(c(-c4ccc5c(c4)OCO5)c2cc1OC)C(=O)OC3]
